(19) United States
(12) Patent Application Publication
(10) Pub. No.: US 2010/0099845 A1
(43) Pub. Date: Apr. 22, 2010

(54) PROTECTED ENANTIOPURE TRIFLUOROTHREONINES AND METHODS OF MAKING AND USING SAME

(75) Inventors: Bruce Yu, Salt Lake City, UT (US); Zhong-Xing Jiang, Salt Lake City, UT (US); Nu Xiao, Salt Lake City, UT (US)

Correspondence Address: Ballard Spahr LLP, SUITE 1000, 999 PEACHTREE STREET, ATLANTA, GA, 30309-3915, US

(73) Assignee: University of Utah Research Foundation, Salt Lake City, UT (US)

(21) Appl. No.: 12/439,328

(22) PCT Filed: Sep. 14, 2007
(86) PCT No.: PCT/US07/78552

§ 371 Date: Dec. 23, 2009

Related U.S. Application Data
(60) Provisional application No. 60/845,228, filed on Sep. 15, 2006.

(51) Int. Cl. C07K 7/08; C07C 59/01; C07K 14/00

(52) U.S. Cl. 530/327; 562/586; 530/300

(57) ABSTRACT

Disclosed are processes for preparing a protected trifluorothreonine, or salt thereof or carboxylate derivative thereof, the process comprising: dihydroxylation of an alkene to yield a dihydroxyl compound; conversion of the dihydroxyl compound to a monohydroxyl compound; protection of the monohydroxyl compound to yield an azide compound; transformation of the azide compound to yield an amino compound; protection of the amino compound to yield a protected amine compound; and oxidation of the protected amine compound to yield the protected trifluorothreonine. Also disclosed are compounds having the structure:

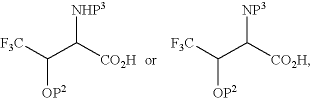

or salt thereof or carboxylate derivative thereof, wherein P² is a hydroxyl protecting group, and wherein P³ is an amine protecting group. This abstract is intended as a scanning tool for purposes of searching in the particular art and is not intended to be limiting of the present invention.

### CROSS-REFERENCE TO RELATED APPLICATION

This application claims the benefit of priority of U.S. Provisional Application No. 60/845,228, filed Sep. 15, 2006, which application is incorporated herein by this reference in its entirety.

### ACKNOWLEDGEMENT

This invention was made with government support under Grants Nos. NIH EB002880 and NIH EB004416 awarded by the National Institutes of Health. The government has certain rights in the invention.

Certain aspects of this work were supported with funding from the Sidney Kimmel Foundation for Cancer Research, wherein Yihua Yu was a Kimmel Scholar.

### BACKGROUND

Peptides and their derivatives are becoming an increasingly important class of pharmaceuticals, both as drugs [Lien, S.; Lowman, H. B. Trends in Biotech. 2003, 21, 556-562.] and as drug delivery vehicles. [Gariépy, J.; Kawamura, K. Trends in Biotech. 2001, 19, 21-28.] Pharmacokinetics, defined as the in vivo absorption, distribution, metabolism, and excretion (ADME) profile of a drug, [Undevia, S. D.; Gomez-Abuin, G.; Ratain, M. J. Nat. Rev. Cancer 2005, 5, 447-458.] can be a factor in determining the efficacy and toxicity of peptide-based pharmaceuticals.

However, conventional peptide chemistry often fails to effectively provide avenues for the tailoring of pharmacokinetics of peptide-based pharmaceuticals. Thus, despite conventional peptide synthetic methodology, there remains a need for methods and compositions that overcome these deficiencies.

In contrast to conventional methods, the incorporation of fluorinated moieties can alter the pharmacokinetic characteristics, and thus the efficacy and toxicity, of peptide-based pharmaceuticals. Further, incorporation of fluorinated moieties can also provide functional groups that can serve as reporters of peptide pharmacokinetics via ¹⁹F MRS.

### SUMMARY

Disclosed are processes for preparing a protected trifluorothreonine having the structure:

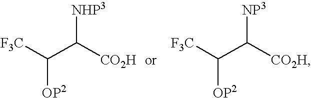

Or a salt thereof or a carboxylate derivative thereof, wherein P² is a hydroxyl protecting group, and wherein P³ is an amine protecting group; the process comprising the steps of: providing an alkene compound having the structure:

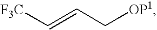

wherein P¹ is a hydroxyl protecting group; dihydroxylation of the alkene compound to yield a dihydroxyl compound having the structure:

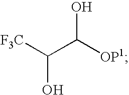

conversion of the dihydroxyl compound to a monohydroxyl compound having the structure:

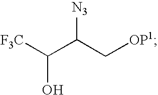

protection of the monohydroxyl compound to yield an azide compound having the structure:

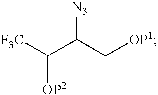

transformation of the azide compound to yield an amino compound having the structure:

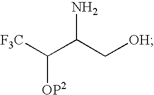

protection of the amino compound to yield a protected amine compound having the structure:

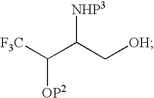

and
oxidation of the protected amine compound to yield the protected trifluorothreonine or the salt thereof or the carboxylate derivative thereof.

Also disclosed are products prepared by a disclosed process.

Also disclosed are compounds having the structure:

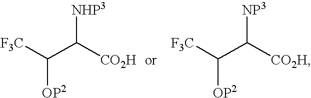

or a salt thereof or a carboxylate derivative thereof, wherein P² is a hydroxyl protecting group, and wherein P³ is an amine protecting group;

Also disclosed are peptides comprising at least one residue of a product of a disclosed process or at least one residue of a disclosed compound.

Also disclosed are pharmaceutical compositions comprising a therapeutically effective amount of one or more compounds comprising a product of a disclosed process, or a residue thereof, or a disclosed compound, or a residue thereof, or a disclosed peptide and a pharmaceutically acceptable carrier for administration in a mammal.

Also disclosed are methods comprising administering an effective amount of one or more compounds comprising the product of a disclosed process, or a residue thereof, or a disclosed compound, or a residue thereof, or a disclosed peptide.

Additional advantages can be set forth in part in the description which follows, and in part can be obvious from the description, or may be learned by practice. Other advantages can be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE FIGURES

The accompanying figures, which are incorporated in and constitute a part of this specification, illustrate several embodiments and together with the description serve to explain the principles of the invention.

FIG. 1 shows structures of four stereoisomers of 4,4,4-trifluorotheonine (tfT) with L-threonine (L-Thr) as reference.

FIG. 2 shows structures of 1a: (2R,3S)-2-(((9H-fluoren-9-yl)methoxy)carbonylamino)-3-tert-butoxy-4,4,4-trifluorobutanoic acid and of 1b: (2S,3R)-2-(((9H-fluoren-9-yl)methoxy)carbonylamino)-3-tent-butoxy-4,4,4-trifluorobutanoic acid

FIG. 3 shows molecular structures of 14a (A) and 14b (B), showing the atom-numbering scheme with 30% thermal ellipsoids.

FIG. 4 shows an HPLC chromatogram of co-injection of 1a, 1b, allo-D-Thr, allo-L-Thr, D-Thr and L-Thr.

### DETAILED DESCRIPTION

Before the present compounds, compositions, articles, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

Although any methods and materials similar or equivalent to those described herein can be used in the practice or testing of the present invention, example methods and materials are now described.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which may need to be independently confirmed.

As used in the specification and the appended claims, the singular forms “a,” “an” and “the” include plural referents unless the context clearly dictates otherwise. Thus, for example, reference to “a compound,” “a polymer,” or “a particle” includes mixtures of two or more such compounds, polymers, or particles, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it can be understood that the particular value forms another embodiment. It can be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that throughout the application, data is provided in a number of different formats and that this data represents endpoints and starting points, and ranges for any combination of the data points.

For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

As used herein, the term “residue” refers to a moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

As used herein, the terms “optional” or “optionally” means that the subsequently described event or circumstance may or may not occur, and that the description includes instances where said event or circumstance occurs and instances where it does not.

As used herein, the term “alkyl” refers to a hydrocarbon group that can be conceptually formed from an alkane, alkene, or alkyne by removing hydrogen from the structure of a cyclic or non-cyclic hydrocarbon compound having straight or branched carbon chains, and replacing the hydrogen atom with another atom or organic or inorganic substituent group. In some aspects of the invention, the alkyl groups are “C₁ to C₆ alkyl” such as methyl, ethyl, propyl, isopropyl, n-butyl, iso-butyl, sec-butyl, tent-butyl, amyl, tent-amyl, and hexyl groups, their alkenyl analogues, their alkynyl analogues, and the like. Many embodiments of the invention comprise “C₁ to C₄ alkyl” groups (alternatively termed “lower alkyl” groups) that include methyl, ethyl, propyl, iso-propyl n-butyl, iso-butyl, sec-butyl, and t-butyl groups, their alkenyl analogues, their alkynyl analogues, or the like. Some of the preferred alkyl groups of the invention have three or more carbon atoms preferably 3 to 16 carbon atoms, 4 to 14 carbon atoms, or 6 to 12 carbon atoms. The alkyl group can be unsubstituted or substituted. A hydrocarbon residue, for example an alkyl group, when described as “substituted,” contains or is substituted with one or more independently selected heteroatoms such as O, S, N, P, or the halogens (fluorine, chlorine, bromine, and iodine), or one or more substituent groups containing heteroatoms (OH, NH₂, NO₂, SO₃H, and the like) over and above the carbon and hydrogen atoms of the substituent residue. Substituted hydrocarbon residues may also contain carbonyl groups, amino groups, hydroxyl groups and the like, or contain heteroatoms inserted into the “backbone” of the hydrocarbon residue. In one aspect, an “alkyl” group can be fluorine substituted. In a further aspect, an “alkyl” group can be perfluorinated.

As used herein, the terms “alkoxy” and “alkoxyl” refer to an —OR radical or group, wherein R is an alkyl radical or group. In one aspect, an “alkoxy” group can be fluorine substituted. In a further aspect, an “alkoxy” group can be perfluorinated.

As used herein, the term “fluorinated” refers to a compound or chemical moiety bearing at least one fluorine atom. That is, at least one hydrogen atom on a moiety has been instead substituted with at least one fluorine atom. One example is a trifluorinated ethyl group, —CH₂CF₃. By “perfluorinated,” it is meant that all hydrogen atoms on a moiety have been instead substituted with fluorine atoms. One example is a perfluorinated methyl group, —CF₃.

As used herein, the term “protecting group” refers to a chemical moiety that temporarily modifies a potentially reactive functional group and protects the functional group from undesired chemical transformations. Protecting group chemistry is known to one of skill in the art. See T. Greene, et al., “Protective Groups in Organic Synthesis,” 2^{nd} ed., Wiley, N.Y., 1991, which is incorporated by reference herein for its teaching of protecting groups and methods of adding and removing protecting groups. Likewise, procedures for removal of the various protecting groups are known to those of skill in the art and are described in various references, including the above-listed “Protective Groups in Organic Synthesis.”

Those of ordinary skill in the art appreciate that certain moieties are incompatible with (i.e., may interfere with) certain chemical transformations as described herein. Thus, it is understood that for certain chemical transformations, certain moieties, e.g., a hydroxyl group or an amino group (primary or secondary), are preferably protected by a suitable protecting group as described herein prior to those transformations. As used herein, the term “protected” refers to a chemical moiety that has been temporarily modified by a protecting group and has been thus protected from undesired chemical transformations. Upon removal of the protecting group (i.e., “deprotection”), the chemical moiety is typically liberated.

As used herein, the term “orthogonal,” when used in connection with protecting groups, refers to the relationship between two or more protecting groups that have mutually exclusive deprotection reaction conditions. That is, one protecting group remains undisturbed under conditions that remove a second protecting group and vice versa. In one aspect, the two or more protecting groups are used to protect two or more chemical moieties having the same chemical structures (e.g., two or more amine moieties). In a further aspect, the two or more protecting groups are used to protect two or more chemical moieties having different chemical structures (e.g., an amine moiety and a carboxylic acid moiety). In a still further aspect, the two or more protecting groups are used to protect one or more chemical moieties having a first chemical structure and one or more chemical moieties having a second chemical structure (e.g., two amine moieties and one carboxylic acid moiety). An example of orthogonal protecting groups is the use of a tent-butyl (tBu) group to protect an alcohol and a 9-fluorenylmethyloxycarbonyl (Fmoc) group to protect an amine.

As used herein, the term “subject” means any target of administration. The subject can be an animal, for example, a mammal (e.g., a human, horse, pig, rabbit, dog, sheep, goat, non-human primate, cow, cat, guinea pig, or rodent), a fish, a bird or a reptile or an amphibian. The term does not denote a particular age or sex. Thus, adult and newborn subjects, as well as fetuses, whether male or female, are intended to be covered. In a further example, the subject can be a human. In an even further example, the subject can be a cell. A “patient” refers to a subject afflicted with a disease or disorder. The term “patient” includes human and veterinary subjects.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. In various aspects, a preparation can be administered therapeutically; that is, administered to treat an existing disease or condition. In further various aspects, a preparation can be administered diagnostically; that is, administered to diagnose an existing disease or condition. In further various aspects, a preparation can be administered prophylactically; that is, administered for prevention of a disease or condition. In a further aspect, “administering” and “administration” can refer to administration to cells that have been removed from a subject (e.g., human or animal), followed by re-administration of the cells to the same, or a different, subject.

As used herein, the term “effective amount” refers to such amount as is capable of performing the function of the compound or property for which an effective amount is expressed. As will be pointed out below, the exact amount required will vary from process to process, depending on recognized variables such as the compounds employed and the processing conditions observed. Thus, it is not typically possible to specify an exact “effective amount.” However, an appropriate effective amount may be determined by one of ordinary skill in the art using only routine experimentation. In various aspects, an amount can be therapeutically effective; that is, effective to treat an existing disease or condition. In further various aspects, a preparation can be prophylactically effective; that is, effective for prevention of a disease or condition.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity may be maintained, for example, by the use of coating materials such as lecithin, by the maintenance of the required particle size in the case of dispersions and by the use of surfactants. These compositions may also contain adjuvants such as preservatives, wetting agents, emulsifying agents, and dispersing agents. Prevention of the action of microorganisms may be ensured by the inclusion of various antibacterial and antifungal agents such as paraben, chlorobutanol, phenol, sorbic acid, and the like. It can also be desirable to include isotonic agents such as sugars, sodium chloride, and the like. Prolonged absorption of the injectable pharmaceutical form may be brought about by the inclusion of agents, such as aluminum monostearate and gelatin, which delay absorption. Injectable depot forms can be made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

Disclosed are the components to be used to prepare the compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions. Thus, if there are a variety of additional steps that can be performed it is understood that each of these additional steps can be performed with any specific embodiment or combination of embodiments of the methods.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. SYMBOLS AND ABBREVIATIONS

Bn: benzyl; Boc: t-butoxycarbonyl; Bz: benzoyl; Cys: cysteine; DCC: 1,3-dicylclohexylcarbodiimide; DEAD: diethylazodicarboxylate; DMAP: 4-dimethylaminopyridine; Fmoc: fluorenylmethoxycarbonyl; HPLC: high-performance liquid chromatography; LC: liquid chromatography; Lys (K): lysine; MRI: magnetic resonance imaging; MRS: magnetic resonance spectroscopy; MS: mass spectrometry; Ms: methanesulfonyl; NMR: nuclear magnetic resonance; Phe: phenylalanine; tBu: t-butyl; TFA: trifluoroacetic acid; tfT: 4,4,4-trifluorothreonine; THF: tetrahydrofuran; Thr: threonine: Trp: tryptophan; and Tyr: tyrosine.

### C. METHODS OF PREPARATION

As described in “Enantioselective Synthesis of (2R,3S)- and (2S,3R)-4,4,4-trifluoror-N-Fmoc-O-tert-butyl-threonin and their racemization-free Incorporation into Oligopeptides via Solid-phase Synthesis” (manuscript accepted to BIOPOLYMERS; http://www3.interscience.wiley.com/cgi-bin/jhome/28380; DOI: 10.1002/bip.20825), which is incorporated herein by reference in its entirety, the present invention addresses the aforementioned deficiencies in conventional peptide chemistry.

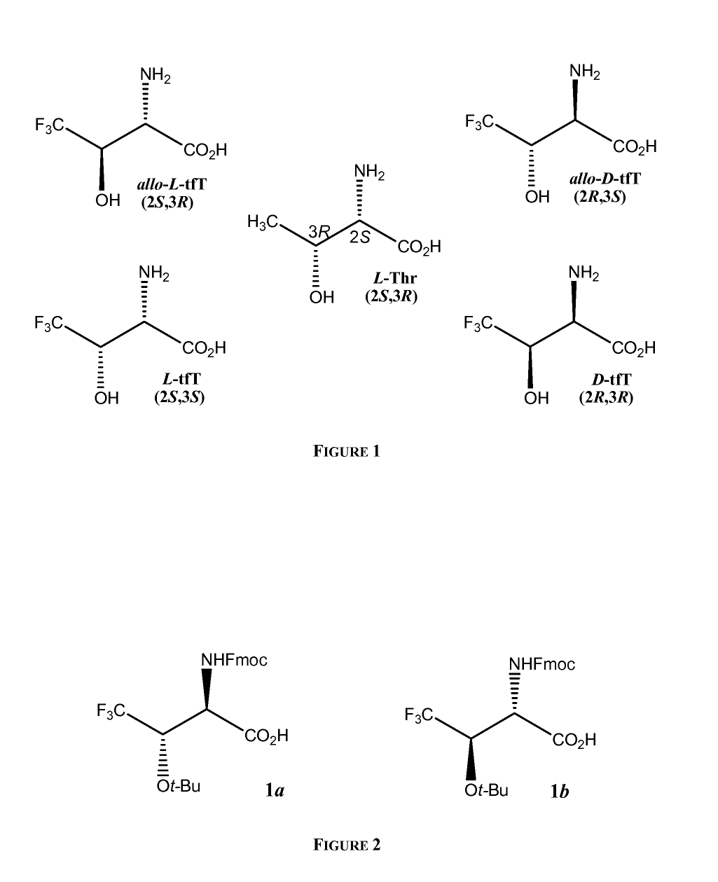

Although both racemic and enantioselective syntheses of free and partially protected 4,4,4-trifluoro-threonine (tfT) have been attempted, [(a).Walborsky, H. M.; Baum, M. E. J. Am. Chem. Soc. 1958, 80, 187-192. (b). Scolastico, C.; Conca, E.; Prati, L.; Guanti, G.; Banfi, L.; Berti, A.; Farina, P.; Valcavi, V. Synthesis 1985, 850-855. (c). Seebach, D.; Juaristi, E.; Miller, D. D.; Schickli, C.; Weber, T. Helv. Chem. Acta 1987, 70, 237-261. (d). Guanti, G.; Banfi, L.; Narisano, E.; Tetrahedron 1988, 44, 5553-5562. (e). Kitazume, T.; Lin, J. T.; Yamazaki, T. Tetrahedron: Asymmetry 1991, 2, 235-238. (f). Von dem Bussche-Hühnefeld, C.; Seebach, D. Chem. Ber. 1992, 125, 1273-1281. (g). Shimizu, M.; Yokota, T.; Fujimori, K.; Fujisawa, T. Tetrahedron: Asymmetry 1993, 4, 835-838. (h). Soloshonok, V. A.; Kukhar, V. P.; Guaiushko, S. V.; Svisttunova, N.Y.; Avilov, D. V.; Kuzmina, N. A.; Reayski, N. I.; Struchkov, Y. T.; Pyrarevshy, A. P.; Belokon, Y. N. J. Chem. Soc., Perkin, Trans. 1 1993, 3143-3155. (i). Soloshonok, V. A.; Hayashi, T.; Ishikawa, K.; Nagashima, N. Tetrahedron Lett. 1994, 35, 1055-1058. (j). Sting, A. R.; Seebach, D. Tetrahedron 1996, 52, 279-290. (k). Jiang, Z.-X.; Qin, Y.-Y.; Qing, F.-L. J. Org. Chem. 2003 68, 7544-7547.] no synthesis of protected tfT in forms suited for solid-phase peptide synthesis (i.e., suitably protected) has been published to date. Without wishing to be bound by theory, it is believed that this is the reason why tfT has not been previously used in peptide research. Since Fmoc solid-phase synthesis is more widely used than its Boc cousin, the synthesis of tfT in the form in which its α-amine is protected by the base-labile Fmoc group while its β-hydroxyl is protected by the acid-labile tert-butyl group (orthogonal protection) was undertaken. The structures of the two example molecules, (2R,3S)-4,4,4-trifluoro-N-Fmoc-O-tent-butyl-threonine (1a) and (2S,3R)-4,4,4-trifluoro-N-Fmoc-O-tert-butyl-threonine (1b), are presented in FIG. 2.

Since 1a and 1b can be used as starting materials for solid-phase peptide synthesis, large quantities are typically needed. Thus, the disclosed methods can use simple and well-established reactions. Also, the disclosed methods can use inexpensive achiral starting materials. Both measures increase the feasibility for the large-scale synthesis of a protected amino acid by those not specialized in chiral organofluorine synthesis.

1. Preparation of Starting Materials

The synthesis of 1a and 1b can start with either 4,4,4-trifluoro-3-oxo-butyric acid ethyl ester 2 or ethyl 4,4,4-trifluoro-crotonate 4. Both 2 and 4 are achiral and relatively inexpensive in the U.S. (currently $0.16/g and $2.30/g, respectively). Since these starting molecules contain the —CF₃ group, use of the trifluoromethylation reagent, FSO₂CF₂CO₂Me, which was used by Qing et al in the synthesis of free tfT, can be avoided. [Jiang, Z.-X.; Qin, Y.-Y.; Qing, F.-L. J. Org. Chem. 2003 68, 7544-7547.] FSO₂CF₂CO₂Me is corrosive and rather expensive in the U.S. (currently $23.50/g). For the construction of two chiral centers, Sharpless asymmetric dihydroxylation (AD) can be utilized. This reaction was selected is because, in one aspect, Sharpless AD can afford high enantioselective synthesis on an industrial scale. [Ahrgren, L.; Sutin, L. Org. Proc. Res. Dev. 1997, 1, 425-427.] Sharpless AD was also the method used by Qing et al. in the synthesis of free tfT. [Jiang, Z.-X.; Qin, Y.-Y.; Qing, F.-L. J. Org. Chem. 2003 68, 7544-7547.] An exemplary synthesis started with the reduction of 2, as illustrated in SCHEME 1.

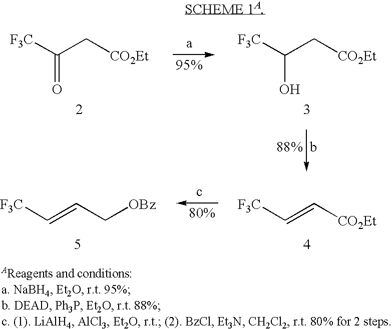

Reduction of ketone 2 with sodium borohydride [Janzen, E. G.; Zhang, Y.-H.; Arimura, M. J. Org. Chem. 1995, 60, 5434-5440.] gave alcohol 3 with a 95% yield which was then treated with triphenyl phosphine and diethylazodicarcarboxylate to afford ethyl 4, 4,4-trifluorocrotonate 4 with an 88% yield. [Bevilacqua, P. F.; Keith, D. D.; Roberts, J. L. J. Org. Chem. 1984, 49, 1430-1434.] Ester 4 then underwent reduction with lithium aluminum hydride in the presence of aluminum chloride [Loh, T.-P.; Li, X.-R. Eur. J. Org. Chem. 1999, 1893-1899.] to give the corresponding alcohol whose hydroxyl group was then protected with benzoyl chloride to give compound 5 with an 80% yield on a 40-gram scale. One reason to employ the benzoyl group (Bz) instead of the benzyl group (Bn) to protect the alcohol is that, in the presence of tert-butyl ether, Bz can be selectively removed in a straightforward method using either hydrolysis or reduction cleavage (step d in SCHEMES 3 & 4). In contrast, attempts to remove Bn in the presence of tert-butyl ether using common hydrogenolysis methods failed.

2. Asymmetric Dihydroxylation

With the trifluoromethylated trans-alkene 5 in hand, Sharpless AD was then employed to construct the two chiral centers simultaneously (SCHEME 2). With a benzoyl protective group on the trifluorinated trans-alkene 5, Sharpless AD on compound 5 proceeded smoothly to give the diol 6a and 6b with excellent yields. When using the benzoyl protecting group, even higher enantioselectivity of 6a and 6b than when using the benzyl analogues was obtained. The enantiomeric excess (e.e.) values of 6a and 6b (without recrystallization) are both over 99% (see Stereochemical characterization). It was also determined that, with Bz as the hydroxyl protective group in 5, the Sharpless AD reaction was completed within 12 hours with excellent yield, even in the absence of methanesulfonamide, a catalyst typically used in Sharpless AD reactions. In contrast, with Bn as the hydroxyl protective group in 5, a much longer reaction time (4 days) was required for the Sharpless AD reaction even in the presence of methanesulfonamide.

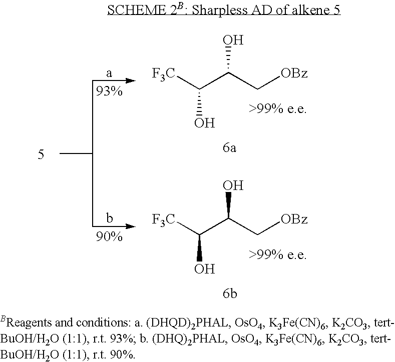

3. Preparation of (2R,3S)-4,4,4-trifluoro-N-Fmoc-O-tert-butyl-Threonine, 1A

From the recrystallized chiral diol 6a, 1a was synthesized over 7 steps (SCHEME 3). Treatment of diol 6a with thionyl chloride and triethyl amine afforded cyclic sulfite intermediate which then underwent oxidation with ruthenium chloride and sodium periodate to give the cyclic sulfate 7a with an 80% yield. Ring opening of the cyclic sulfate 7a with sodium azide followed by hydrolysis of the resulted sulfonic acid provided alcohol 8a with a 98% yield.

When protecting the hydroxyl group in alcohol 8a with the tent-butyl group, two early attempts were unsuccessful. In the first attempt, a mixture of alcohol 8a and liquid iso-butylene was treated with phosphoric acid and boron trifluoride at −70° C. [Micheli, R. A.; Hajos, Z. G.; Cohen, N.; Parrish, D. R.; Louis, A.; Sciamanna, P. W.; Scott, M. A.; Wehrli, P. A. J. Org. Chem. 1975, 40, 675-681.] The mixture was then stirred for 3 days at room temperature. Rather than leading to 9a, this resulted in the recovery of the starting material 8a. In the second attempt, the method recently reported by Bartoli for the preparation of tert-butyl ethers with tert-butyl dicarbonate was used. [Bartoli, G.; Bosco, M.; Locatelli, M.; Marcantoni, E.; Melchiorre, P.; Sambri, L. Org. Lett. 2005, 7, 427-430.] However, after two days, this also resulted in the recovery of the starting material 8a. Fortunately, treatment of 8a with iso-butylene in the presence of Amberlyst® 15 ion-exchange resin [Alexakis, A.; Duffault, J. M. Tetrahedron Lett. 1988, 29, 6243-6246.]resulted in partial conversion of alcohol 8a to tert-butyl ether 9a. When sulfuric acid [Zhang, X. G.; Ni, W. J.; van der Donk, W. A. J. Org. Chem. 2005, 70, 6685-6692.] was employed to catalyze this reaction with liquid iso-butylene in a sealed vessel, the tert-butyl ether product 9a was isolated with a 70% yield. Without wishing to be bound by theory, it is believed that the low reactivity of alcohol 8a is likely due to both the electron-withdrawing effect of —CF₃ group, which makes the adjacent hydroxyl group more acidic and less nucleophilic, and the bulkiness of —CF₃, which can hinder the attack of the alcohol by in situ generated tert-butyl cation.

With the tert-butyl ether 9a in hand, removal of the benzoyl protective group was then undertaken. In order to avoid racemization, a reaction with very mild condition (diisobutylaluminum hydride reduction) was employed, and alcohol 10a was isolated with a 93% yield. The successful removal of the benzoyl group under mild conditions is another benefit brought by the replacement of benzyl with benzoyl in 5. Alcohol 10a was then subjected to palladium-catalyzed hydrogenation of its azido group to give the amine 11a with an 89% yield. Protection of the amino group with FmocCl (9-fluorenylmethyl chloroformate) yielded alcohol 12a with a 98% yield which then underwent Jones oxidation to afford the final product 1a in a 91% yield on a multi-gram scale.

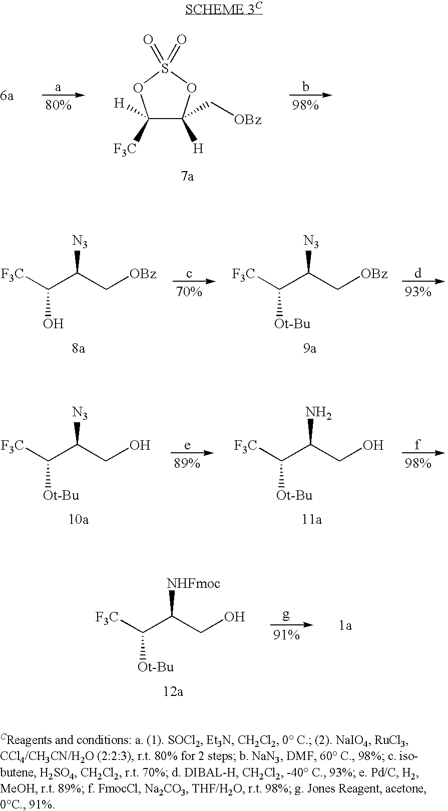

4. Preparation of (2S,3R)-4,4,4-trifluoro-N-Fmoc-O-tert-butyl-threonine, 1B

By employing the same procedure for the synthesis of 1a, 1b was synthesized from recrystallized chiral diol 6b on a multi-gram scale (SCHEME 4).

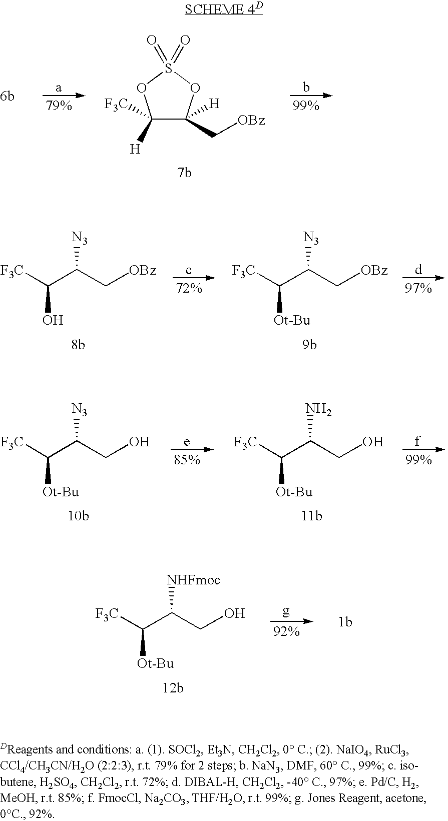

While the synthetic routes discussed above can be performed as solution-phase syntheses, which involves the synthesis of compounds in individual reaction vessels, other methods can be performed. For example, combinatorial based syntheses or solid phase syntheses can be used and will depend on the particular compounds to be synthesized, the availability of reagents, or preference.

a. Processes

In one aspect, the invention relates to a process for preparing a protected trifluorothreonine having the structure:

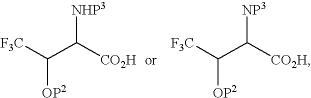

or a salt thereof or a carboxylate derivative thereof, wherein P² is a hydroxyl protecting group, and wherein P³ is an amine protecting group; the process comprising the steps of: providing an alkene compound having the structure:

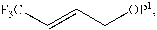

wherein P¹ is a hydroxyl protecting group; dihydroxylation of the alkene compound to yield a dihydroxyl compound having the structure:

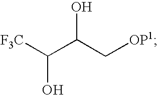

conversion of the dihydroxyl compound to a monohydroxyl compound having the structure:

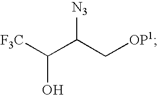

protection of the monohydroxyl compound to yield an azide compound having the structure:

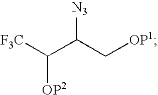

transformation of the azide compound to yield an amino compound having the structure:

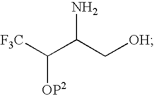

protection of the amino compound to yield a protected amine compound having the structure:

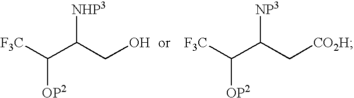

and
oxidation of the protected amine compound to yield the protected trifluorothreonine or the salt thereof or the carboxylate derivative thereof.

P¹ and P² can be, independently, hydroxyl protecting groups known to those of skill in the art. Suitable hydroxyl protecting groups include, but are not limited to, esters, allyl ethers, ethers, silyl ethers, alkyl ethers, arylalkyl ethers, and alkoxyalkyl ethers. Examples of such esters include formates, acetates, carbonates, and sulfonates. Specific examples include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate(trimethylacetyl), crotonate, 4-methoxy-crotonate, benzoate, p-benzylbenzoate, 2,4,6-trimethylbenzoate, carbonates such as methyl, 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl. Examples of such silyl ethers include trimethylsilyl, triethylsilyl, t-butyldimethylsilyl, t-butyldiphenylsilyl, triisopropylsilyl, and other trialkylsilyl ethers. Alkyl ethers include methyl, benzyl, p-methoxybenzyl, 3,4-dimethoxybenzyl, trityl, t-butyl, allyl, and allyloxycarbonyl ethers or derivatives. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyranyl ethers. Examples of arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, 2- and 4-picolyl. In a further aspect, P¹ is benzoyl. In a further aspect, P² is t-butyl.

P³ can be an amine protecting group known to those of skill in the art. Suitable amine protecting groups, taken with the amino moiety to which it is attached, include, but are not limited to, aralkylamines, carbamates, allyl amines, amides, and the like. Examples of such groups include t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, benzyl (Bn), fluorenylmethylcarbonyl (Fmoc), acetyl, chloroacetyl, dichloroacetyl, trichloroacetyl, phenylacetyl, trifluoroacetyl, benzoyl, and the like. In further aspects, an amine protecting group is acetyl, chloroacetyl, dichloroacetyl, trichloroacetyl, phenylacetyl, or trifluoroacetyl. In still other embodiments, an amine group can be in protected form as a phthalimide or azide.

In a further aspect, P² and P³ are orthogonal protecting groups. For example, P² can be tent-butyl, and P³ can be 9-fluorenylmethyloxycarbonyl.

It is also understood that the methods can provide a protected trifluorothreonine or a salt thereof or a carboxylate derivative thereof. That is, in one aspect, the carboxylic acid moiety can be optionally provided as a salt thereof. Suitable salts include monovalent, divalent, and trivalent salts. Monovalent salts include salts prepared with monovalent cations, including ammonium salts, quaternary amine salts, lithium salts, sodium salts, potassium salts, and the like. Divalent salts include salts prepared with divalent cations, including beryllium salts, magnesium salts, calcium salts, and the like. Trivalent salts include salts prepared with trivalent cations, including aluminum salts, iron salts, Ln(III) salts, and the like.

It is also understood that, in one aspect, the carboxylic acid moiety can be optionally provided as a carboxylate derivative (i.e, a protected carboxylate) thereof. Suitable carboxylate protecting groups include, but are not limited to, esters, including substituted or unsubstituted C₁₋₆ aliphatic esters, optionally substituted aryl esters, silyl esters, activated esters, amides, hydrazides, and the like. Examples of such ester groups include methyl, ethyl, propyl, isopropyl, butyl, isobutyl, tert-butyl, benzyl, and phenyl wherein each group is optionally substituted.

Said another way, in one aspect, the carboxylic acid structure is intended to include salts thereof and derivatives thereof, as disclosed herein, as well as equivalents thereof.

b. Providing Step

In a further aspect, the providing step comprises the steps of: reducing a ketone having the structure:

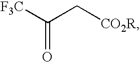

wherein R is an alkyl group, to yield an alcohol having the structure:

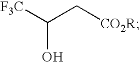

eliminating the hydroxyl group of the alcohol to yield an acrylate having the structure:

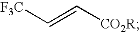

and
reducing the acrylate and protecting the product to yield an alkene compound having the structure:

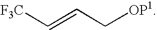

It is also understood that the providing step can comprise one or more chemical reactions known to those of skill in the art of organic chemical synthesis, provided the reactions yield an alkene compound having the structure:

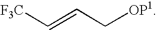

In a further aspect, the providing step comprises obtaining the alkene commercially.

c. Dihydroxylation Step

In a further aspect, the dihydroxylation step is asymmetric dihydroxylation. For example, the dihydroxylation step can comprise treatment of the alkene compound with (DHQD)₂PHAL and OsO₄ or treatment with (DHQ)₂PHAL and OsO₄. In a still further aspect, the dihydroxylation step is performed in the substantial absence of methanesulfonamide.

In a further aspect, the dihydroxyl compound has the structure:

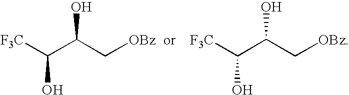

d. Conversion Step

In one aspect, the conversion step comprises the steps of: reacting the dihydroxyl compound with a thionyl halide, followed by treatment with NaIO₄ and RuCl₃; and reacting the product of step (a) with NaN₃. The monohydroxyl compound, in one aspect, has the structure:

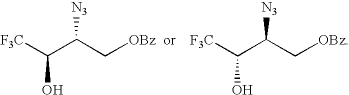

In a further aspect, the protection of the monohydroxyl compound step comprises treatment with isobutene.

In one aspect, the azide compound has the structure:

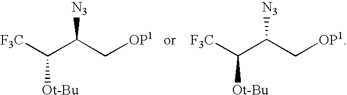

e. Transformation Step

In one aspect, the transformation step comprises the steps of: treatment with DIBAL-H; and catalytic hydrogenation. In a further aspect, the amino compound has the structure:

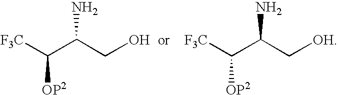

The protection of the amino compound step can comprise treatment with FmocCl. The protected amine compound can, for example, have the structure:

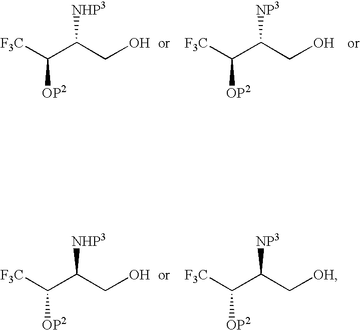

f. Oxidation Step

It is understood that oxidation reactions known to those of skill in the art can be employed in the oxidation step, provided the reaction is capable of converting an alcohol (e.g., primary alcohol) to a carboxylic acid or a salt thereof or a derivative thereof. In one aspect, the oxidation step comprises treatment with Jones Reagent.

In a further aspect, the protected trifluorothreonine has the structure:

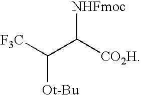

It is understood that, in one aspect, the disclosed methods can be used to provide the disclosed compounds.

5. Stereochemical Characterization

The stereochemistry of the synthesis was verified at three stages. Firstly, right after constructing the chiral centers, the enantiomeric purity of the Sharpless AD products 6a and 6b was verified. Direct determination of the enantiomeric excess (e.e.) values for 6a and 6b by chiral chromatography was unsuccessful (Three different chiral columns, ChiraDex, Chirobiotic T and Ultron ES-Pepsin, were tried, each failing to resolve 6a and 6b). Instead, 6a and 6b were transformed into their Mosher esters 13a (over 99% yield) and 13b (over 99% yield), respectively (Scheme 5). As 13a and 13b are diastereoisomers, their ¹⁹F NMR signals are distinct. The e.e. values of 6a and 6b were inferred from the diastereomeric excess (d.e.) values of 13a and 13b determined by ¹⁹F NMR spectroscopy. From the d.e. values of 13a and 13b, the inferred e.e. values of 6a and 6b are over 99%.

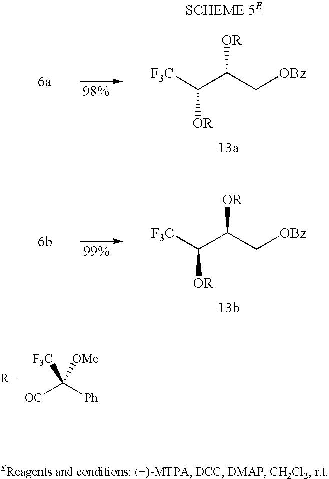

Secondly, the enantiomeric purity of the final products 1a and 1b (after silica gel column purification) was determined directly by analytical chiral chromatography. [The HPLC conditions are as follows: column, ChiraDex (250 mm×4.6 mm I.D., 5 μm, Agilent Technologies); mobile phase, 5 mM NaH₂PO4-Na₂HPO₄ in MeOH—H₂O (60/40, v/v, pH=5.6); flow rate, 0.60 mL/min; temperature, 10° C. Samples were purified by silica-gel column chromatography and dissolved in mobile phase before injection.] High e.e. values were achieved for both enantiomers: 96.8% for 1a and >99% for 1b. After recrystallization, the e.e. values of 1a and 1b were both higher than 99.5%.

Thirdly, to determine the absolute configurations of both series of chiral molecules, amino alcohols 11a and 11b were transformed to their camphor-sulfonamides 14a and 14b respectively (SCHEME 6) for X-ray crystallographic analysis. Treatment of 11a with (1R)-(−)-camphor-10-sulfonyl chloride in the presence of 4-dimethylaminopyridine (DMAP) afforded camphorsulfonamide 14a with a 48% yield. In the same way, camphor-sulfonamide 14b was isolated with a 52% yield after treating 11b with (1S)-(+)-camphor-10-sulfonyl chloride. So a pair of enantiomers, 14a and 14b, were obtained. During the workup, small amounts of 15a and 15b were also isolated with a 15% yield and a 13% yield, respectively.

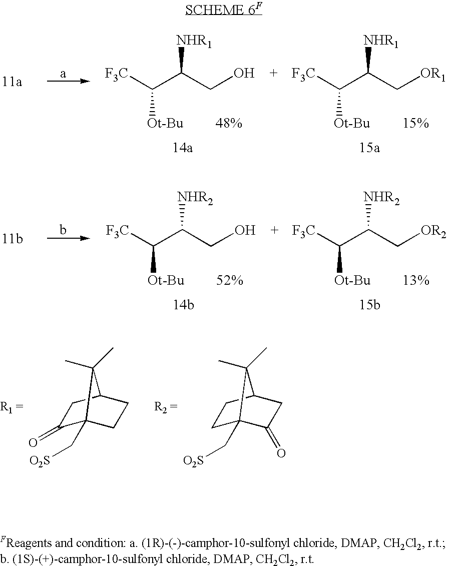

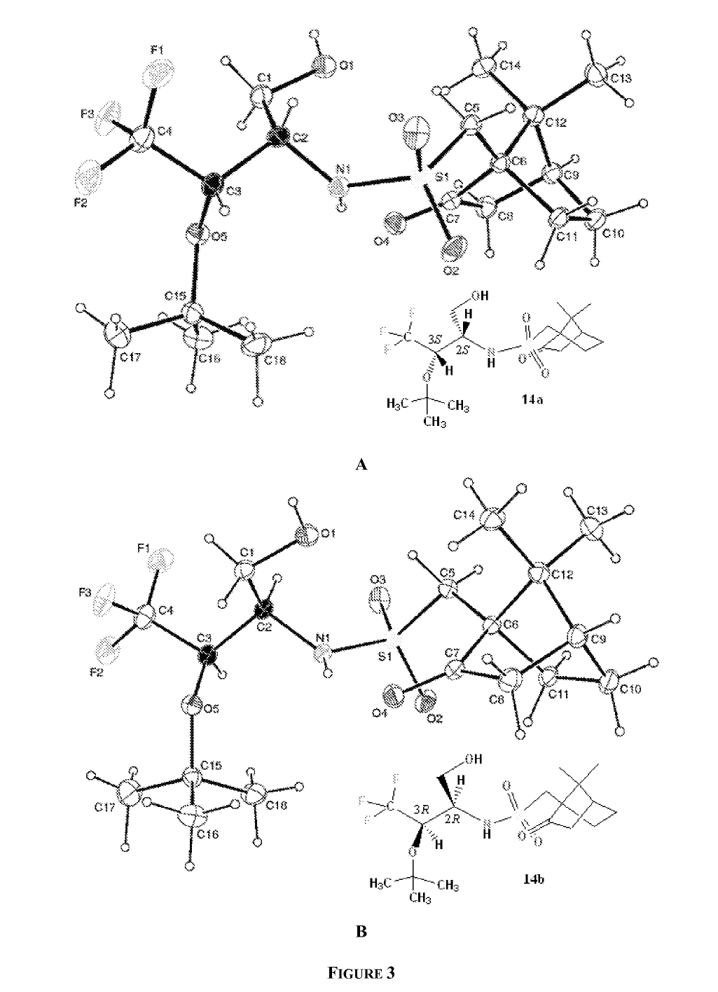

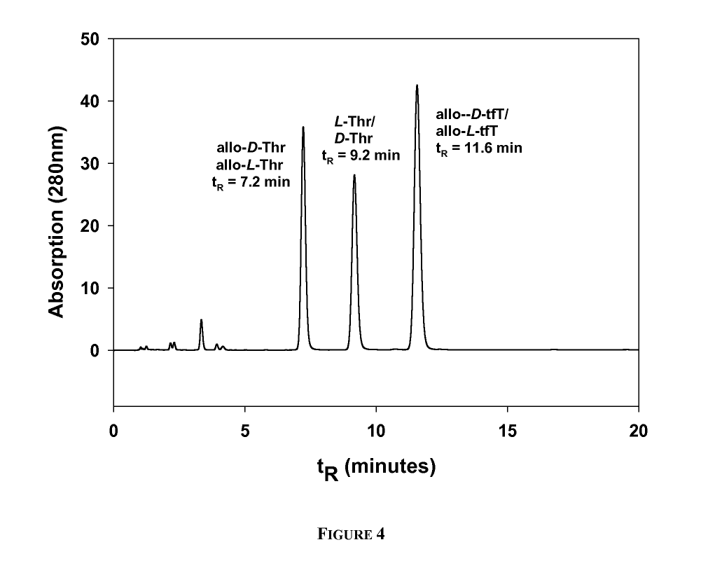

Single crystals of compound 14a and 14b were collected by slow evaporation of their respective CH₂Cl₂/hexane solutions. With the aid of single crystal X-ray diffraction, the absolute configurations of compound 14a and 14b were determined to be (2S,3S) and (2R,3R) respectively (FIG. 3). Therefor, Sharpless AD reaction of alkene 5 with (DHQD)₂PHAL gives the chiral diol 6a with the (2R,3S) configuration, while that with (DHQ)₂PHAL gives the chiral diol 6b with the (2S,3R) configuration.

6. Hydrophobicity Fmoc-tfT vs. Fmoc-Thr

As fluorination can decrease the hydrophilicity of a molecule [(a) Böhm, H-J.; Banner, D.; Bendels, S.; Kansy, M.; Kuhn, B.; Müller, K.; Obst-Sander, U.; Stahl, M. Chem BioChem. 2004, 5, 637-643. (b) Schweizer, E.; Hoffmann-Röder, A.; Schärer, K.; Olsen, J. A.; Fäh, C.; Seiler, P.; Obst-Sander, U.; Wagner, B.; Kansy, M.; Diederich, F. ChemMedChem, 2006, 1, 611-621.] and hence reduce membrane permeability [Abbruscato, T. J.; Williams, S. A.; Misicka, A., Lipkowski, A. W., Hryby, V. J.; Davis, T. P. J. Pharmacol. Exp. Therapeut. 1996, 276, 1049-1057.], the hydrophobicity of Fmoc-tfT (1a and 1b) relative to their non-fluorinated counterparts was determined. The chromatography method developed by Hodges and coworkers [Kovacs, J. M.; Mant, C. T.; Hodges, R. S. Biopolymers (Peptide Science) 2006, 84, 283-297.], who determined the relative hydrophobicity of 23 L-amino acids and their D-enantiomers, was used. As pointed out by Hodges and coworkers [Kovacs, J. M.; Mant, C. T.; Hodges, R. S. Biopolymers (Peptide Science) 2006, 84, 283-297.], a criterion for measuring true hydrophobicity of an amino acid is that the D/L enantiomers should give the same retention time, t_{R}. FIG. 4 shows the co-injection of 1a (denoted as allo-D-tfT), 1b (denoted as allo-L-tfT), their non-fluorinated counterparts: (2R,3R)-N-Fmoc-O-tert-butyl-threonine (denoted as allo-D-Thr), (2S,3S)-N-Fmoc-O-tent-butyl-threonine (denoted as allo-L-Thr), as well as the other two isomers of Fmoc-protected threonine: (2R,3S)-N-Fmoc-O-tert-butyl-threonine (denoted as D-Thr) and (2S,3R)-N-Fmoc-O-tent-butyl-threonine (denoted as L-Thr). All of the enantiomeric pairs, allo-D-Thr/allo-L-Thr (t_{R}=7.2 min), D-Thr/L-Thr (t_{R}=9.2 min) and allo-D-tfT/allo-L-tfT (t_{R}=11.6 min), co-elute, satisfying the criterion established by Hodges and coworkers. [Kovacs, J. M.; Mant, C. T.; Hodges, R. S. Biopolymers (Peptide Science) 2006, 84, 283-297.] In reversed-phase chromatography, a larger retention time typically indicates a more hydrophobic molecule. The allo-D-tfT/allo-L-tfT pair is more retentive than the allo-D-Thr/allo-L-Thr pair (Δt_{R}=11.6−7.2=4.2 min). This indicates that replacing the —CH₃ group in Thr by —CF₃ in tfT indeed renders the molecule more hydrophobic. To put matters into perspective, the retention time difference between allo-D-tfT/allo-L-tfT and allo-D-Thr/allo-L-Thr (Δt_{R}=4.2 min) is larger than that between Ala and Gly (Δt_{R}=2.8 min), comparable to that between Cys and Ala (Δt_{R}=4.8 min), and smaller than that between Val and Ala (Δt_{R}=10.6 min). [Kovacs, J. M.; Mant, C. T.; Hodges, R. S. Biopolymers (Peptide Science) 2006, 84, 283-297.]

In FIG. 4, all chromatography runs followed exactly the same conditions used by Hodges and coworkers [Zhang, X. G.; Ni, W. J.; van der Donk, W. A. J. Org. Chem. 2005, 70, 6685-6692.]. The HPLC conditions are as follows: column, Kromasil C18 (150 mm×2.1 mm I.D., 5 μm, 100 Å pore size, Higgins Analytical, Inc., CA); mobile phase, A: 0.2% TFA (trifluoroacetic acid) in water, B: 0.2% TFA in ACN (acetonitrile); condition, linear AB gradient (0.25% ACN/min, starting from 55% B); flow rate, 0.3 mL/min; temperature, 25° C. 1a and 1b were purified by silica-gel column chromatography and dissolved in mobile phase B before injection. allo-D-N-Fmoc-O-tert-butyl-Thr and allo-L-N-Fmoc-O-tert-butyl-Thr were from BACHEM California Inc. D-N-Fmoc-O-tert-butyl-Thr and L-N-Fmoc-O-tert-butyl-Thr were from Novabiochem.

### D. METHODS OF USING

As an analog of the proteinogenic amino acid threonine (Thr), 4,4,4-trifluorothreonine (tfT) can be incorporated either inside or outside the receptor-binding site of a peptide drug (e.g., Thr⁶ and Thr⁸ lie inside and outside, respectively, of the peptide drug octreotide, which has the sequence D-Phe¹-Cys²-Phe³-D-Trp⁴-Lys⁵-Thr⁶-Cys⁷-Thr⁸-ol). [Rueter, J. K.; Mattern, R.-H.; Zhang, L.; Taylor, J.; Morgan, B.; Hoyer, D.; Goodman, M. Biopolymers, 2000, 53, 497-505.] One benefit of incorporating a trifluoromethylated amino acid into a receptor-binding site of a peptide drug is that the —CF₃ group can report the receptor binding event via ¹⁹F MRS analysis. [(a). Gerig, J. T.; Klinkenborg, J. C.; J. Am. Chem. Soc. 1980, 102, 4267-4268. (b). Jenkins, B. G.; Lauffer, R. B. Mol. Pharmacol. 1990, 37, 111-118. (c). Dalvit, C.; Ardini, E.; Fogliatto, G. P.; Mongelli, N.; Veronesi, M. Drug Discovery Today 2004, 9, 595-602.]

Fluorinated amino acids as pharmacokinetics modulators can present two advantages: enhancement of membrane permeability (particularly to increase the crossing of the blood-brain barrier) and increase in the in vivo half-life (t_{1/2}) of peptide drugs, such as that based on octreotide, for, e.g., the diagnosis and treatment of brain illness. Currently, octreotide-based drugs need to be administered into the brain in a locoregional fashion using a stereotactically inserted port-a-cath and have a t_{1/2} of ca. 2 hours. [(a). Merlo, A.; hausmann, O.; Wasner, M.; Steiner, P.; Otte, A.; Jermann, E.; Freitag, P.; Reubi, J.-C.; Muller-Brand, J.; Gratzl, O.; Macke, H. R. Clin. Cancer Res. 1999, 5, 1025-1033. (b). Schumacher, T.; Hofer, S.; Eichhorn, K.; Wasner, M.; Zimmerer, S.; Freitag, P.; Probst, A.; Gratzl, O.; Reubi, J.-C.; Maecke, H. R.; Mueller-Brand, J.; Merlo, A. Eur. J. Nucl. Med. 2002, 29, 486-493. (c). Arnold, R.; Simon, B.; Wied, M. Digestion, 2000, 62(suppl 1), 84-91.]

In one aspect, the methods relate to the treatment of a disease or condition. For example, the methods can relate to administering an effective amount of one or more compounds comprising the product of a disclosed process, or a residue thereof, or a disclosed compound, or a residue thereof, or a disclosed peptide to a subject. In a further aspect, the methods can further comprise the step of detecting fluorine using, for example, ¹⁹F NMR.

### E. COMPOUNDS

Like threonine (Thr), tfT has two chiral carbons (C2 and C3) and hence four stereoisomers: (2S,3R), (2R,3S), (2S,3S) and (2R,3R), corresponding to allo-L-Thr, allo-D-Thr, L-Thr and D-Thr, respectively (FIG. 1). Since the chirality of permeability enhancers can affect membrane permeation of chiral drugs, [Kommuru, T. R.; Khan, M. A.; Reddy, I. Chirality 1999, 11, 536-540.] different stereoisomers of tfT allow one to explore the impact exerted by the chirality of fluorinated amino acids on peptide membrane permeability. Of the four stereoisomers of tfT, the synthesis of (2R,3S) and (2S,3R) isomers in their protected forms for Fmoc-solid-phase peptide synthesis are described herein as examples.

In one aspect, the invention relates to compounds having the structure:

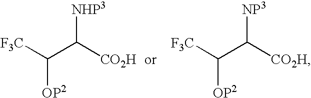

or a salt thereof or a carboxylate derivative thereof, wherein P² is a hydroxyl protecting group, and wherein P³ is an amine protecting group.

P² can be a hydroxyl protecting group known to those of skill in the art. Suitable hydroxyl protecting groups include, but are not limited to, esters, allyl ethers, ethers, silyl ethers, alkyl ethers, arylalkyl ethers, and alkoxyalkyl ethers. Examples of such esters include formates, acetates, carbonates, and sulfonates. Specific examples include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate(trimethylacetyl), crotonate, 4-methoxy-crotonate, benzoate, p-benzylbenzoate, 2,4,6-trimethylbenzoate, carbonates such as methyl, 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl. Examples of such silyl ethers include trimethylsilyl, triethylsilyl, t-butyldimethylsilyl, t-butyldiphenylsilyl, triisopropylsilyl, and other trialkylsilyl ethers. Alkyl ethers include methyl, benzyl, p-methoxybenzyl, 3,4-dimethoxybenzyl, trityl, t-butyl, allyl, and allyloxycarbonyl ethers or derivatives. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyranyl ethers. Examples of arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, 2- and 4-picolyl. In a further aspect, P' is benzoyl. In a further aspect, P² is t-butyl.

P³ can be an amine protecting group known to those of skill in the art. Suitable amine protecting groups, taken with the amino moiety to which it is attached, include, but are not limited to, aralkylamines, carbamates, allyl amines, amides, and the like. Examples of such groups include t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, benzyl (Bn), fluorenylmethylcarbonyl (Fmoc), acetyl, chloroacetyl, dichloroacetyl, trichloroacetyl, phenylacetyl, trifluoroacetyl, benzoyl, and the like. In further aspects, an amine protecting group is acetyl, chloroacetyl, dichloroacetyl, trichloroacetyl, phenylacetyl, or trifluoroacetyl. In still other embodiments, an amine group can be in protected form as a phthalimide or azide.

In a further aspect, P² and P³ are orthogonal protecting groups. For example, P² can be tent-butyl, and P³ can be 9-fluorenylmethyloxycarbonyl.

It is also understood that the compounds can be provided as a protected trifluorothreonine or a salt thereof or a carboxylate derivative thereof. That is, in one aspect, the carboxylic acid moiety can be optionally provided as a salt thereof. Suitable salts include monovalent, divalent, and trivalent salts. Monovalent salts include salts prepared with monovalent cations, including ammonium salts, quaternary amine salts, lithium salts, sodium salts, potassium salts, and the like. Divalent salts include salts prepared with divalent cations, including beryllium salts, magnesium salts, calcium salts, and the like. Trivalent salts include salts prepared with trivalent cations, including aluminum salts, iron salts, Ln(III) salts, and the like.

It is also understood that, in one aspect, the carboxylic acid moiety can be optionally provided as a carboxylate derivative (i.e, a protected carboxylate) thereof. Suitable carboxylate protecting groups include, but are not limited to, esters, including substituted or unsubstituted C₁₋₆ aliphatic esters, optionally substituted aryl esters, silyl esters, activated esters, amides, hydrazides, and the like. Examples of such ester groups include methyl, ethyl, propyl, isopropyl, butyl, isobutyl, tert-butyl, benzyl, and phenyl wherein each group is optionally substituted.

Said another way, in one aspect, the carboxylic acid structure is intended to include salts thereof and derivatives thereof, as disclosed herein, as well as equivalents thereof.

In a further aspect, the invention relates to compounds having the structure:

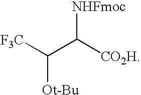

For example, the compound can have the structure:

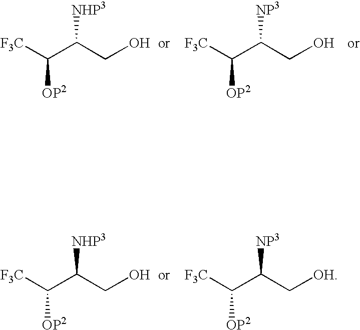

In a further aspect, the invention relates to (2R,35)-2-(((9H-fluoren-9-yl)methoxy)carbonylamino)-3-tert-butoxy-4,4,4-trifluorobutanoic acid and/or (2S,3R)-2-(((9H-fluoren-9-yl)methoxy)carbonylamino)-3-tert-butoxy-4,4,4-trifluorobutanoic acid

It is understood that, in one aspect, the disclosed compounds can be prepared from the disclosed methods.

### F. COMPOSITIONS

In one aspect, the invention relates to a pharmaceutical composition comprising a therapeutically effective amount of one or more compounds comprising the product of a disclosed process, or a residue thereof, or a disclosed compound, or a residue thereof, or a disclosed peptide and a pharmaceutically acceptable carrier for administration in a mammal.

In one aspect, the compositions can relate to peptides. In a further aspect, the invention can relate to a peptide comprising at least one residue of the product of a disclosed process or at least one residue of a disclosed compound.

In a further aspect, the peptide can have the structure:

Z-D-Phe-Cys-M-D-Trp-Lys-A-Cys-A-X,

wherein each A independently comprises a residue of threonine or a residue of the product of a disclosed process or a residue of a disclosed compound; wherein M comprises Phe or

Tyr or a derivative thereof (i.e., a substituted Phe or Tyr, for example, iodinated Tyr or nitrated Tyr); wherein X comprises a terminal end group selected from carboxyl, ester, amide, and alcohol; wherein Z comprises a terminal end group selected from amino, formyl, acetyl, and succinyl.

In a further aspect, the peptide can have the structure:

Z-D-Phe-Cys-M-D-Trp-Lys-A-Cys-Thr-X,

wherein A is a residue of the product of a disclosed process or a residue of a disclosed compound.

In a further aspect, the peptide can have the structure:

Z-D-Phe-Cys-M-D-Trp-Lys-Thr-Cys-A-X,

wherein A is a residue of the product of a disclosed process or a residue of a disclosed compound.

In a further aspect, the peptide can have the structure:

Z-D-Phe-Cys-M-D-Trp-Lys-A-Cys-A-X,

wherein each A independently comprises a residue of the product of a disclosed process of any or a residue of a disclosed compound.

### G. KITS

Disclosed herein are kits that are drawn to compounds and/or reagents that can be used in practicing the methods disclosed herein. The kits can include any reagent or combination of reagents discussed herein or that would be understood to be required or beneficial in the practice of the disclosed methods. For example, the kits could include reagents to perform complexation reactions discussed in certain embodiments of the methods, as well as buffers and solvents required to use the reagents as intended.

### H. COMPOSITIONS WITH SIMILAR FUNCTIONS

It is understood that the compositions disclosed herein have certain functions. Disclosed herein are certain structural requirements for performing the disclosed functions, and it is understood that there are a variety of structures which can perform the same function which are related to the disclosed structures, and that these structures will ultimately achieve the same result.

### I. EXPERIMENTAL

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Synthesis of Benzoic Acid (E)-4,4,4-trifluoro-but-2-enyl ester, 5

To a suspension of anhydrous AlCl₃ (34.1 g, 0.26 mol) in diethyl ether (80 mL) at 0° C. was added a solution of LiAlH₄ (28.5 g, 0.75 mol) in diethyl ether (500 mL). The resulting mixture was then stirred at this temperature for 15 min. A solution of compound 4 (52.0 g, 0.31 mol) in diethyl ether (40 mL) was added at 0° C. and stirring was continued for another 4 h. Then at 0° C., water (28 mL), NaOH aqueous solution (5.7 g NaOH in 57 mL water) and another portion of water (86 mL) were added in sequence slowly to the reaction mixture. The resulting solution was filtered and condensed under normal pressure. The residue was dissolved in a solution of pyridine (54.0 mL, 0.67 mol) and CH₂Cl₂ (800 mL). Then benzoyl chloride (65.0 mL, 0.56 mol) was added dropwise at 0° C. The resulting solution was stirred overnight at room temperature. Then the reaction mixture was washed with 2 N HCl aqueous solution. The organic phase was collected and the aqueous phase was extracted with ether (3×100 mL). The combined organic phase was washed with brine, dried over MgSO₄ and concentrated under vacuum. The residue was purified by column chromatography on silica gel (hexane:ethyl acetate=30:1) to give a colorless oil (57.2 g, 80%). ¹H NMR (CDCl₃, 400 MHz): δ 7.97-7.30 (m, 5H), 6.44 (m, 1H), 5.87 (m, 1H), 4.83 (m, 2H); ¹⁹F NMR (CDCl₃, 376.4 MHz): δ-67.24 (d, J=6.1 Hz).

### 2. Synthesis of (2R,3S)-Benzoic Acid 4,4,4-trifluoro-2,3-dihydroxy-butyl ester, 6A

To a stirred mixture of tent-butyl alcohol (400 mL) and water (400 mL) were added (DHQD)₂PHAL (1.3 g, 1.67 mmol), K₃Fe(CN)₆ (81.5 g, 247 mmol), K₂CO₃ (34.2 g, 247 mmol), and OsO₄ (6.5 mL of 0.1 M aqueous solution, 0.65 mmol) at room temperature. After the solid was dissolved, the solution was cooled to 0° C. Olefin 5 (19.0 g, 82.5 mmol) was added in one portion, and the heterogeneous slurry was stirred vigorously at room temperature overnight. Then Na₂SO₃ (81 g, 643 mmol) was added to the resulting yellow solution. The mixture was stirred for 30 min and its color turned into dark brown. The upper organic phase was collected. The lower aqueous solution was extracted with ethyl acetate (5×150 mL). The combined organic phase was washed with saturated KHSO₄ aqueous solution (100 mL) and saturated K₂SO₄ aqueous solution (100 mL) to recover some of (DHQD)₂PHAL. Then the organic solution was dried over anhydrous MgSO₄, filtered and concentrated in vacuum. The residue was purified by flash chromatography on silica gel (hexane:ethyl acetate=5:1) to give 6a as a white solid (20.2 g, 93% yield). 6a was recrystallized from hexane and CH₂Cl₂ to achieve higher purity: [α]²⁰_{D}=−12.7 (c 0.82, CHCl₃); mp=96° C.; ¹H NMR (CDCl₃, 400 MHz): δ 8.05-7.46 (m, 5H), 4.48 (m, 2H), 4.37 (m, 1H), 4.03 (m, 1H), 3.24 (d, J=8.8 Hz, 1H), 2.80 (d, J=5.2 Hz, 1H); ¹⁹F NMR (CDCl₃, 376.4 MHz): δ −79.83 (d, J=5.2 Hz); ¹³C NMR (CDCl₃, 100.4 MHz): δ 167.0, 133.9, 130.0, 129.4, 128.8, 124.6 (q, J=283.2 Hz), 69.5 (q, J=30.1 Hz), 67.0, 65.4; MS (CI): m/z 265 (M⁺+1, 100); HRMS (CI): Calcd for C₁₁H₁₂F₃O₄ 265.0688, found 265.0674.

### 3. Synthesis of (4R,5S)-Benzoic Acid 2,2-dioxo-5-trifluoromethyl-2λ⁶-1,3,2-dioxathiolan-4-ylmethyl ester, 7A

To a solution of recrystallized diol 6a (10.0 g, 37.9 mmol) and triethylamine (15.3 g, 151.6 mmol) in CH₂Cl₂ (200 mL) was slowly added thionyl chloride (9.0 g, 75.8 mmol) at 0° C. over 20 min. The reaction mixture was stirred for another 60 min at 0° C. and then diluted with cold ether (100 mL). Then cold water (100 mL) was added to the resulting deep brown organic solution. The organic phase was collected and the aqueous phase was extracted with cold ether. The combined organic phase was washed with cold brine and dried over anhydrous MgSO₄. After removing solvent below 30° C., the residue was purified by a short pad of silica gel to give the cyclic sulfite. The cyclic sulfite was then dissolved in water (90 mL), CH₃CN (60 mL) and CCl₄ (60 mL). Then NaIO₄ (9.7 g, 45.5 mmol) and RuCl₃ (20 mg) were added to the solution and the resulting mixture was vigorously stirred for 2 h at room temperature. Ether (100 mL) and saturated NaHCO₃ solution (100 mL) were added to the reaction mixture. The organic phase was collected and the aqueous phase was extracted with ether. The combined organic phase was washed with brine, dried over anhydrous MgSO₄ and concentrated in vacuo. The residue was purified by flash chromatography on silica gel (hexane:ethyl acetate=10:1) to give 7a as a white solid (9.8 g, 80%). [α]²⁰_{D}=+5.0 (c 0.98, CHCl₃); mp=57° C.; ¹H NMR (CDCl₃, 400 MHz): δ 8.08-7.47 (m, 5H), 5.29 (m, 1H), 5.12 (m, 1H), 4.74 (dd, J=12.8, 4.0 Hz, 2H); ¹⁹F NMR (CDCl₃, 376.4 MHz): δ −79.72 (d, J=6.8 Hz); ¹³C NMR (CDCl₃, 100.4 MHz): δ 165.8, 134.3, 130.2, 129.0, 128.4, 121.4 (q, J=280.2 Hz), 77.8, 75.9 (q, J=36.1 Hz), 61.7; MS (CI): m/z 327 (M⁺+1, 100); HRMS (CI): Calcd for C₁₁H₉F₃O₆S 326.0072, found 326.0074.

### 4. Synthesis of (2S,3S)-Benzoic Acid 2-azido-4,4,4-trifluoro-3-hydroxy-butyl ester, 8A

The solution of cyclic sulfate 7a (7.7 g, 23.6 mmol) and sodium azide (3.1 g, 47.2 mmol) in DMF (100 mL) was stirred for 4 h at 80° C. The solvent was carefully removed by distillation under reduced pressure below 80° C. Then THF (200 mL), water (1.0 mL), and sulfuric acid (3.0 mL, 96%) were added. The resulting suspension was stirred for 1 h and NaHSO₃ solid was then added. The reaction mixture was stirred for additional 20 min and filtered through a pad of silica gel. The filtrate was concentrated in vacuum and the residue was purified by column chromatography on silica gel (hexane:ethyl acetate=8:1) to give 8a as a white solid (6.7 g, 98%). [α]²⁰_{D}+20.9 (c 1.83, CHCl₃); mp=74° C.; ¹H NMR (CDCl₃, 400 MHz): δ 7.94-7.32 (m, 5H), 4.63 (m, 1H), 4.52 (m, 1H), 4.08 (m, 1H), 3.97 (m, 1H), 3.88 (m, 1H); ¹⁹F NMR (CDCl₃, 367.4 MHz): δ −78.85 (d, J=5.1 Hz); ¹³C NMR (CDCl₃, 100.4 MHz): δ 167.2, 134.0, 130.1, 129.1, 128.9, 124.3 (q, J=282.5 Hz), 70.1 (q, J=28.1 Hz), 64.2, 59.7; MS (CI): m/z 290 (M⁺+1, 100); HRMS (CI): Calcd for C₁₁H₁₁F₃N₃O₃ 290.0753, found 290.0742.

### 5. Synthesis of (2S,3S)-Benzoic Acid 2-azido-3-tert-butoxy-4,4,4-trifluoro-butyl ester, 9A

To a solution of compound 8a (23.0 g, 80 mmol) in anhydrous CH₂Cl₂ (300 mL) was added liquid isobutylene (100 mL) and H₂SO₄ (1.0 mL, 96%) at −30° C. The resulting mixture was stirred for 4 days at room temperature in a sealed vessel. After releasing the pressure slowly, saturated Na₂CO₃ aqueous solution was added and the resulted mixture was stirred for an additional 10 min. The organic phase was collected and the aqueous phase was extracted with CH₂Cl₂. The combined organic phase was dried over anhydrous Na₂SO₄ and concentrated under vacuum. The residue was purified by column chromatography on silica gel (hexane:ethyl acetate=20:1) to give 9a as a yellow oil (19.0 g, 70%) and recovered 8a (6.0 g). [α]²⁰_{D}−1.8 (c 1.44, CHCl₃); ¹H NMR (CDCl₃, 400 MHz): δ 8.07-7.45 (m, 5H), 4.70 (dd, J=12.0, 3.2 Hz, 1H), 4.40 (dd, J=12.0, 8.8 Hz, 1H), 4.10 (m, 2H), 1.20 (s, 9H); ¹⁹F NMR (CDCl₃, 367.4 MHz): δ −76.49 (d, J=7.3 Hz); ¹³C NMR (CDCl₃, 100.4 MHz): δ 166.3, 133.6, 130.0, 129.6, 128.8, 124.1 (q, J=289.2 Hz), 78.0, 71.7 (q, J=30.8 Hz), 64.3, 60.9, 28.5; MS (CI): m/z 346 (M⁺+1, 100); HRMS (CI): Calcd for C₁₅H₁₉F₃N₃O₃ 346.1379, found 346.1393.

### 6. (2S,3S)-2-azido-3-tert-butoxy-4,4,4-trifluoro-butan-1-ol, 10A

Compound 9a (14.0 g, 41 mmol) was dissolved in anhydrous CH₂Cl₂ (200 mL) and the solution was then cooled to −70° C. Diisobutylaluminum hydride (110 mL 1M solution in hexane) was added drop wise and the resulted mixture was stirred at −40° C. for 30 min. Then 100 mL ethyl acetate was added. After stirring for another 30 min at room temperature, 1 N HCl aqueous solution was added. The organic phase was collected and the aqueous phase was extracted with ether. The combined organic phase was washed with brine and dried over anhydrous Na₂SO₄. After concentrated under vacuum, the residue was purified by column chromatography on silica gel (hexane:ethyl acetate=4:1) to give 10a as a colorless oil (9.0 g, 93%). [α]²⁰_{D}+25.3 (c 1.46, CHCl₃); ¹H NMR (CDCl₃, 400 MHz): δ 4.02 (m, 1H), 3.81-3.68 (m, 3H), 2.54 (b, 1H), 1.21 (s, 9H); ¹⁹F NMR (CDCl₃, 367.4 MHz): δ −76.08 (d, J=6.3 Hz); ¹³C NMR (CDCl₃, 100.4 MHz): δ 124.3 (q, J=283.8 Hz), 78.1, 71.8 (q, J=29.5 Hz), 63.2, 61.9, 28.3; MS (CI): m/z 242 (M⁺+1, 100); HRMS (CI): Calcd for C₈H₁₅F₃N₃O₂242.1117, found 242.1114.

### 7. Synthesis of (2S,3S)-2-amino-3-tert-butoxy-4,4,4-trifluoro-butan-1-ol, 11A

Compound 10a (8.0 g, 33 mmol) was dissolved in methanol (200 mL) and 10% Pd/C powder (1.0 g) was added. This mixture was stirred overnight under a H₂ atmosphere at room temperature. After filtration and condensed under vacuum, the residue was purified by column chromatography on silica gel (ethyl acetate:methanol=4:1) to give 11a as a colorless oil (6.4 g, 89%). [α]²⁰_{D}=−4.5 (c 1.23, CHCl₃); ¹H NMR (CDCl₃, 400 MHz): δ 3.90 (m, 1H), 3.58 (m, 2H), 3.02 (m, 1H), 2.19 (b, 3H), 1.19 (s, 9H); ¹⁹F NMR (CDCl₃, 367.4 MHz): δ −73.92 (d, J=7.7 Hz); ¹³C NMR (CDCl₃, 100.4 MHz): δ 124.9 (q, J=283.8 Hz), 77.3, 72.7 (q, J=25.4 Hz), 63.0, 53.9, 28.4; MS (CI): m/z 216 (M⁺+1, 100); HRMS (CI): Calcd for C₈H₁₇F₃NO₂ 216.1212, found 216.1206.

### 8. Synthesis of (2S,3S)-(2-tert-butoxy-3,3,3-trifluoro-1-hydroxymethyl-propyl)-carbamic acid 9H-fluoren-9-ylmethyl ester, 12A

Compound 11a (6.0 g, 28 mmol) was dissolved in THF (200 mL) and H₂O (200 ml). FmocCl (11.0 g, 42 mmol) and NaHCO₃ (7.0 g, 84 mmol) were added at 0° C. and the mixture were stirred for 4 h at room temperature. Then the reaction mixture was extracted with ethyl acetate and the combined organic phase was dried over anhydrous MgSO₄. After concentrated under vacuum, the residue was purified by column chromatography on silica gel (hexane:ethyl acetate=4:1) to give 12a as a white solid (12.0 g, 98%). [α]²⁰_{D}=−11.5 (c 1.05, CHCl₃); mp=121° C.; ¹H NMR (CDCl₃, 400 MHz): δ 7.77-7.29 (m, 8H), 5.71 (d, J=6.8 Hz, 1H), 4.52-4.41 (m, 2H), 4.32 (m, 1H), 4.20 (m, 1H), 4.12 (m, 1H), 3.87 (m, 1H), 3.64 (m, 1H), 2.72 (b, 1H), 1.19 (s, 9H); ¹⁹F NMR (CDCl₃, 367.4 MHz): δ −75.17 (d, J=6.6 Hz); ¹³C NMR (CDCl₃, 100.4 MHz): δ 156.1, 144.0, 143.9, 141.6, 128.0, 127.3, 125.2, 125.1, 124.3 (q, J=284.8 Hz), 120.3, 78.1, 73.0 (q, J=28.1 Hz), 67.1, 62.2, 50.8, 47.4, 28.5; MS (CI): m/z 438 (M⁺+1, 78); HRMS (CI): Calcd for C₂₃H₂₇F₃NO₄ 438.1893, found 438.1910.

### 9. Synthesis of (2R,3S)-3-tert-butoxy-2-(9H-fluoren-9-ylmethoxycarbonylamino)-4,4,4-trifluoro-butyric acid, 1A

Compound 12a (5.0 g 11.5 mmol) was dissolved in acetone (80 mL) and Jones reagent (11.9 mL, 6.2 N aqueous solution, 71.0 mmol) was added drop wise at 0° C.

The brown solution was stirred at 0° C. for 2 h. Then iso-propanol (60 mL) was added slowly and the mixture was stirred for an additional 10 min. After removal the solvent, the residue was dissolved in water (250 mL) and extracted with ethyl acetate. The combined organic layer was dried over anhydrous MgSO₄ and condensed under vacuum. The residue was purified by column chromatography on silica gel (ethyl acetate:methanol=9:1) to give 1a as a white solid (4.7 g, 91%). 1a was recrystallized from hexane and CH₂Cl₂ to achieve higher purity: [α]²⁰_{D}=+7.4, (c 0.79, CHCl₃); mp=161° C.; ¹H NMR (CD₃OD, 400 MHz): δ 7.77-7.28 (m, 8H), 4.66 (m, 2H), 4.40 (m, 1H), 4.21 (m, 2H), 3.34 (s, 1H), 1.26 (s, 9H); ¹⁹F NMR (CD₃OD, δ67.4 MHz): δ −73.16 (d, J=5.1 Hz); ¹³C (CD₃OD, 100.4 MHz): δ 173.7, 157.4, 144.1, 144.0, 141.3, 127.6, 127.0, 125.3, 125.1, 125.0 (q, J=283.2 Hz), 119.7, 76.9, 70.9 (q, J=28.4 Hz), 67.1, 57.6, 29.7, 27.0; MS (CI): m/z 452 (M′+1, 25); HRMS (CI): Calcd for C₂₃H₂₅F₃NO₅ 452.1686, found 452.1676.

### 10. Synthesis of (2R,3S)-benzoic acid 4,4,4-trifluoro-2,3-bis-((R)-3,3,3-trifluoro-2-methoxy-2-phenyl-propionyloxy)-butyl ester, 13A

Compound 6a (27 mg, 0.1 mmol), DCC(N,N-dicyclohexyl-carbodiimde) (63 mg 0.3 mmol) and DMAP (4 mg) were dissolved in CH₂Cl₂ (3 mL). (+)-MTPA ((R)-(+)-α-methoxy-α-(trifluoromethyl)phenylacetic acid) (72 mg, 0.3 mmol) was added to the solution. The resulting mixture was stirred at room temperature for 16 h (An ¹⁹F NMR spectrum was then recorded with 0.5 mL of the reaction mixture and the d.e. value was found to be over 99%, based on ¹⁹F signals of fluorine atoms at the C4 position). The reaction mixture was concentrated in vacuum. The residue was purified by flash chromatography on silica gel (hexane:ethyl acetate=5:1) to give 13a as a colorless oil (70 mg, over 99%). [α]²⁰_{D}+35.7 0.91, CHCl₃); ¹H NMR (CDCl₃, 400 MHz): δ 7.98-7.26 (m, 15H), 5.99 (m, 1H), 5.87 (m, 1H), 4.37 (dd, J=12.0, 7.6 Hz, 1H), 4.27 (dd, J=12.0, 6.0 Hz, 1H), 3.46 (s, 3H), 3.44 (s, 3H); ¹⁹F NMR (CDCl₃, 376.4 MHz): 6-74.51 (s, 3F), −74.67 (s, 3F), −76.25 (d, J=6.4 Hz, 3F); ¹³C NMR (CDCl₃, 100.4 MHz): δ 165.5, 165.3, 139.3, 136.7, 133.7, 131.0, 130.4, 130.2, 129.8, 128.8, 128.6, 128.5, 127.4, 127.3, 123.0 (q, J=288.7 Hz), 122.8 (q, J=287.9 Hz), 120.0 (q, J=281.7 Hz), 85.1 (q, J=29.1 Hz), 68.5 (q, J=32.9 Hz), 68.1, 61.2, 55.5, 55.4, 29.7; MS (CI): m/z: 697 (M+1, 7.8); HRMS (CI): Calcd for C₃₁H₂₆F₉O₈ 697.1485, found 697.1467.

### 11. Synthesis of the Reaction of Compound 11A with (1R)-(−)-camphor-10-sulfonyl chloride to give 14A and 15A

Compound 11a (701 mg, 3.3 mmol) and DMAP (400 mg, 3.3 mmol) were dissolved in anhydrous CH₂Cl₂ (20 mL). (1R)-(−)-camphor-10-sulfonyl chloride (840 mg, 3.3 mmol) was then added at 0° C. The resulted solution was stirred overnight at room temperature and washed with 2 N HCl aqueous solution. Organic phase was collected and the aqueous phase was extracted with ether. The combined organic solvent were washed with brine and dried over anhydrous MgSO₄. After concentrated under vacuum, the residue was purified by column chromatography on silica gel (hexane:ethyl acetate=5:1) to give 14a as a white solid (671 mg, 48%), 15a as a white solid (314 mg, 15%) and recovered some compound 11a (161 mg).

Compound 14a: [α]²⁰_{D}=−17.5 (c 1.03, CHCl₃); mp=168° C.; ¹H NMR (CDCl₃, 400 MHz): δ 5.93 (d, J=6.8 Hz, 1H), 4.40 (m, 1H), 4.01 (m, 1H), 3.80 (m, 2H), 3.60 (AB, J=15.2 Hz, 1H), 2.99 (AB, J=15.2 Hz, 1H), 2.52 (dd, J=8.8, 3.2 Hz, 1H), 2.42 (m, 1H), 2.19 (m, 2H), 2.05 (m, 2H), 1.56 (s, 1H), 1.47 (m, 1H), 1.31 (s, 9H), 1.02 (s, 3H), 0.90 (s, 3H); ¹⁹F NMR (CDCl₃, 367.4 MHz): δ −75.47 (d, J=7.7 Hz); ¹³C (CDCl₃, 100.4 MHz): δ 217.4, 124.2 (q, J=284.0 Hz), 77.9, 74.5 (q, J=29.1 Hz), 61.6, 59.4, 55.1, 51.0, 48.9, 43.0, 42.7, 28.5, 27.1, 26.6, 19.9, 19.4; MS (CI) m/z: 430 (M⁺+1, 75); HRMS (CI) Calcd for C₁₈H₃₁F₃NO₅S 430.1876, found 430.1870.

Compound 15a: [α]²⁰_{D}=−23.3 (c 0.95, CHCl₃); mp=144° C.; ¹H NMR (CDCl₃, 400 MHz): δ 6.08 (d, J=6.4 Hz, 1H), 4.55 (dd, J=10.8, 7.2 Hz, 1H), 4.35 (m, 2H), 4.12 (m, 1H), 3.57 (dd, J=14.8, 3.6 Hz, 2H), 3.02 (dd, J=18.4, 14.8 Hz, 2H), 2.41 (m, 3H), 2.13 (m, 3H), 2.03 (m, 5H), 1.68 (m, 1H), 1.46 (m, 2H), 1.32 (s, 9H), 1.08 (s, 3H), 1.02 (s, 3H), 0.94 (s, 3H), 0.88 (s, 3H); ¹⁹F NMR (CDCl₃, 367.4 MHz): δ −75.65 (d, J=7.7 Hz); ¹³C (CDCl₃, 100.4 MHz): δ 216.7, 215.2, 124.0 (q, J=284.8 Hz), 77.7, 73.3 (q, J=29.1 Hz), 68.3, 59.5, 58.0, 54.3, 51.9, 48.9, 48.4, 46.9, 43.0, 42.9, 42.7, 42.6, 29.7, 28.5, 27.0, 26.9, 24.8, 20.0, 19.7, 19.6, 19.5; MS (CI) m/z: 644 (M⁺+1, 3.8); HRMS (CI) Calcd for C₂₈H₄₅F₃NO₈S₂ 644.2539, found 644.2521.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A process for preparing a protected trifluorothreonine having the structure: 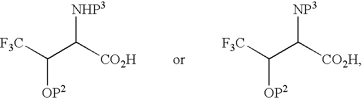
or a salt thereof or a carboxylate derivative thereof,
wherein P² is a hydroxyl protecting group, and
wherein P³ is an amine protecting group;
the process comprising the steps of
a) providing an alkene compound having the structure: 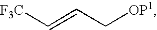
wherein P¹ is a hydroxyl protecting group;
b) dihydroxylation of the alkene compound to yield a dihydroxyl compound having the structure: 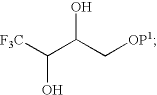
c) conversion of the dihydroxyl compound to a monohydroxyl compound having the structure: 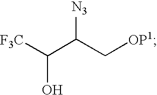
d) protection of the monohydroxyl compound to yield an azide compound having the structure: 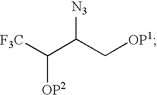
e) transformation of the azide compound to yield an amino compound having the structure: 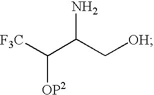
f) protection of the amino compound to yield a protected amine compound having the structure: 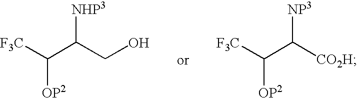
and
g) oxidation of the protected amine compound to yield the protected trifluorothreonine or the salt thereof or the carboxylate derivative thereof.

2-5. (canceled)

6. The process of claim 1, wherein the providing step comprises the steps of:
a) reducing a ketone having the structure: 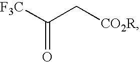
wherein R is an alkyl group,
to yield an alcohol having the structure: 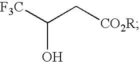
b) eliminating the hydroxyl group of the alcohol to yield an acrylate having the structure: 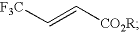
and
c) reducing the acrylate and protecting the product to yield an alkene compound having the structure:

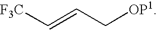

7. The process of claim 1, wherein the dihydroxylation step is asymmetric dihydroxylation.

8-9. (canceled)

10. The process of claim 1, wherein the dihydroxyl compound has the structure:

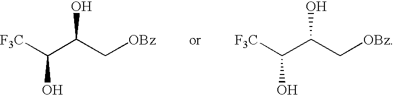

11. The process of claim 1, wherein the conversion step comprises the steps of:
a) reacting the dihydroxyl compound with a thionyl halide, followed by treatment with NaIO₄ and RuCl₃; and
b) reacting the product of step (a) with NaN₃.

12. The process of claim 1, wherein the monohydroxyl compound has the structure:

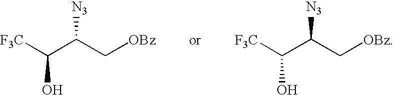

13. The process of claim 1, wherein the protection of the monohydroxyl compound step comprises treatment with isobutene.

14. The process of claim 1, wherein the azide compound has the structure:

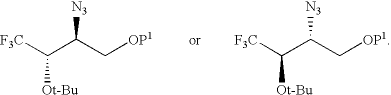

15. The process of claim 1, wherein the transformation step comprises the steps of:
a) treatment with DIBAL-H; and
b) catalytic hydrogenation.

16. The process of claim 1, wherein the amino compound has the structure:

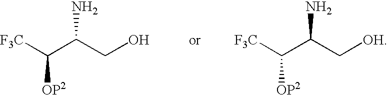

17. (canceled)

18. The process of claim 1, wherein the protected amine compound has the structure:

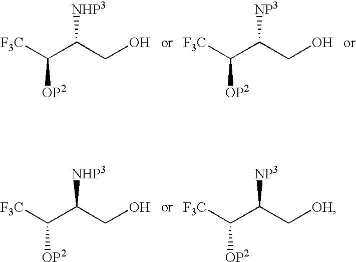

19. The process of claim 1, wherein the protected trifluorothreonine has the structure:

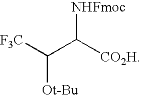

20. (canceled)

21. A compound having the structure: 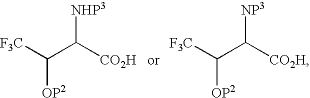
or a salt thereof or carboxylate derivative thereof,
wherein P² is a hydroxyl protecting group, and
wherein P³ is an amine protecting group.

22-24. (canceled)

25. The compound of claim 21, having the structure:

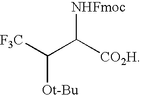

26. The compound of claim 21, having the structure:

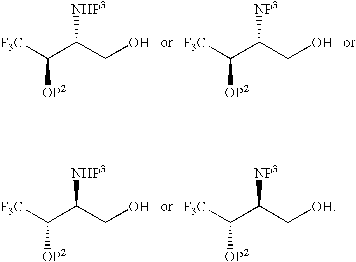

27. (2R,3S)-2-(((9H-fluoren-9-yl)methoxy)carbonylamino)-3-tert-butoxy-4,4,4-trifluorobutanoic acid.

28. (2S,3R)-2-(((9H-fluoren-9-yl)methoxy)carbonylamino)-3-tert-butoxy-4,4,4-trifluorobutanoic acid

29. (canceled)

30. A peptide comprising at least one residue of the product of a process for preparing a protected trifluorothreonine having the structure: 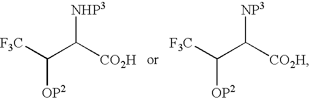
or a salt thereof or a carboxylate derivative thereof,
wherein P² is a hydroxyl protecting group, and
wherein P³ is an amine protecting group;
the process comprising the steps of:
a) providing an alkene compound having the structure: 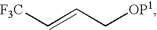
wherein P¹ is a hydroxyl protecting group;
b) dihydroxylation of the alkene compound to yield a dihydroxyl compound having the structure: 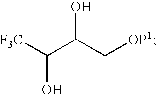
c) conversion of the dihydroxyl compound to a monohydroxyl compound having the structure: 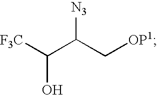
d) protection of the monohydroxyl compound to yield an azide compound having the structure: 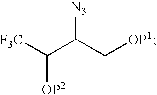
e) transformation of the azide compound to yield an amino compound having the structure: 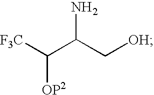
f) protection of the amino compound to yield a protected amine compound having the structure: 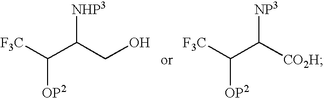
and
g) oxidation of the protected amine compound to yield the protected trifluorothreonine or the salt thereof or the carboxylate derivative thereof, or
at least one residue of a compound having the structure: 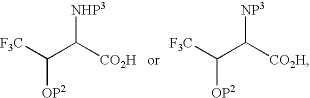
or a salt thereof or carboxylate derivative thereof,
wherein P² is a hydroxyl protecting group, and
wherein P³ is an amine protecting group.

31. The peptide of claim 30, having the structure:
Z-D-Phe-Cys-M-D-Trp-Lys-A-Cys-A-X,
wherein each A independently comprises a residue of threonine or
a residue of a process for preparing a protected trifluorothreonine having the structure: 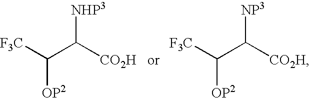
or a salt thereof or a carboxylate derivative thereof,
wherein P² is a hydroxyl protecting group, and
wherein P³ is an amine protecting group;
the process comprising the steps of:
a) providing an alkene compound having the structure: 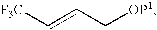
wherein P¹ is a hydroxyl protecting group;
b) dihydroxylation of the alkene compound to yield a dihydroxyl compound having the structure: 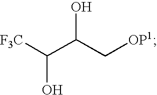
c) conversion of the dihydroxyl compound to a monohydroxyl compound having the structure: 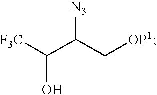
d) protection of the monohydroxyl compound to yield an azide compound having the structure: 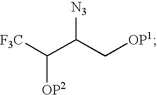
e) transformation of the azide compound to yield an amino compound having the structure: 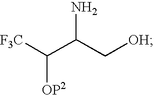
f) protection of the amino compound to yield a protected amine compound having the structure: 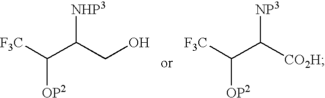
and
g) oxidation of the protected amine compound to yield the protected trifluorothreonine or the salt thereof or the carboxylate derivative thereof, or
a residue of a compound having the structure: 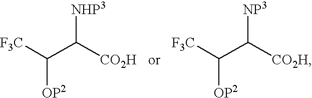
a compound having the structure:
or a salt thereof or carboxylate derivative thereof,
wherein P² is a hydroxyl protecting group, and
wherein P³ is an amine protecting group;
wherein M comprises Phe or Tyr or a derivative thereof;
wherein X comprises a terminal end group selected from carboxyl, ester, amide, and alcohol;
wherein Z comprises a terminal end group selected from amino, formyl, acetyl, and succinyl.

32. The peptide of claim 31, having the structure:
Z-D-Phe-Cys-M-D-Trp-Lys-A-Cys-Thr-X,
wherein A is a residue of the product of a process for preparing a protected trifluorothreonine having the structure: 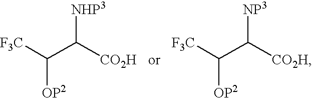
or a salt thereof or a carboxylate derivative thereof,
wherein P² is a hydroxyl protecting group, and
wherein P³ is an amine protecting group;
the process comprising the steps of:
a) providing an alkene compound having the structure: 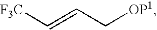
wherein P¹ is a hydroxyl protecting group;
b) dihydroxylation of the alkene compound to yield a dihydroxyl compound having the structure: 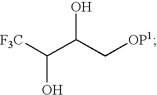
c) conversion of the dihydroxyl compound to a monohydroxyl compound having the structure: 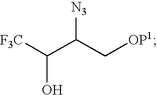
d) protection of the monohydroxyl compound to yield an azide compound having the structure: 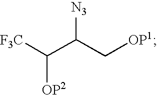
e) transformation of the azide compound to yield an amino compound having the structure: 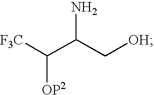
f) protection of the amino compound to yield a protected amine compound having the structure: 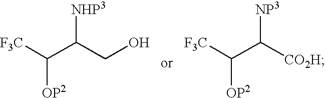
and
g) oxidation of the protected amine compound to yield the protected trifluorothreonine or the salt thereof or the carboxylate derivative thereof, or
a residue of a compound having the structure: 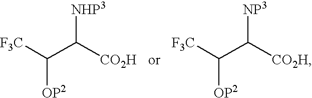
or a salt thereof or carboxylate derivative thereof,
wherein P² is a hydroxyl protecting group, and
wherein P³ is an amine protecting group.

33. The peptide of claim 31, having the structure:
Z-D-Phe-Cys-M-D-Trp-Lys-Thr-Cys-A-X,
wherein A is a residue of the product of a process for preparing a protected trifluorothreonine having the structure: 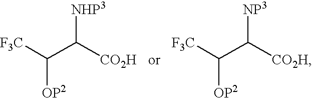
or a salt thereof or a carboxylate derivative thereof,
wherein P² is a hydroxyl protecting group, and
wherein P³ is an amine protecting group;
the process comprising the steps of:
a) providing an alkene compound having the structure: 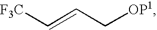
wherein P¹ is a hydroxyl protecting group;
b) dihydroxylation of the alkene compound to yield a dihydroxyl compound having the structure: 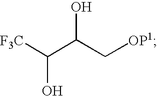
c) conversion of the dihydroxyl compound to a monohydroxyl compound having the structure: 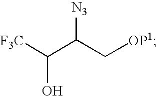
d) protection of the monohydroxyl compound to yield an azide compound having the structure: 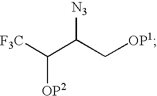
e) transformation of the azide compound to yield an amino compound having the structure: 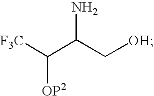
f) protection of the amino compound to yield a protected amine compound having the structure: 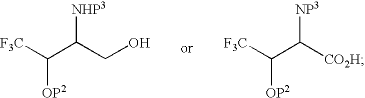
and
g) oxidation of the protected amine compound to yield the protected trifluorothreonine or the salt thereof or the carboxylate derivative thereof, or
a residue of a compound having the structure: 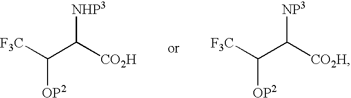
or a salt thereof or carboxylate derivative thereof,
wherein P² is a hydroxyl protecting group, and
wherein P³ is an amine protecting group.

34. The peptide of claim 31, having the structure:
Z-D-Phe-Cys-M-D-Trp-Lys-A-Cys-A-X,
wherein each A independently comprises a residue of the product of a process for preparing a protected trifluorothreonine having the structure: 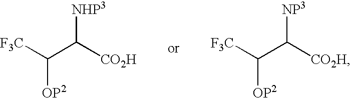
or a salt thereof or a carboxylate derivative thereof,
wherein P² is a hydroxyl protecting group, and
wherein P³ is an amine protecting group;
the process comprising the steps of:
a) providing an alkene compound having the structure: 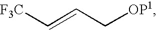
wherein P¹ is a hydroxyl protecting group;
b) dihydroxylation of the alkene compound to yield a dihydroxyl compound having the structure: 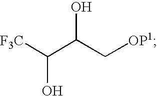
c) conversion of the dihydroxyl compound to a monohydroxyl compound having the structure: 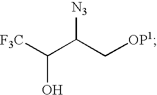
d) protection of the monohydroxyl compound to yield an azide compound having the structure: 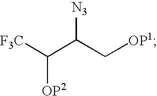
e) transformation of the azide compound to yield an amino compound having the structure: 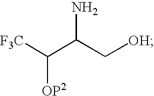
f) protection of the amino compound to yield a protected amine compound having the structure: 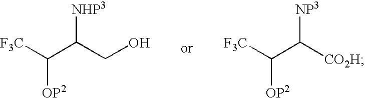
and
g) oxidation of the protected amine compound to yield the protected trifluorothreonine or the salt thereof or the carboxylate derivative thereof, or
a residue of a compound having the structure: 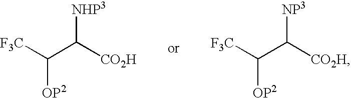
or a salt thereof or carboxylate derivative thereof,
wherein P² is a hydroxyl protecting group, and
wherein P³ is an amine protecting group.

35-38. (canceled)
